(19) United States
(12) Patent Application Publication
(10) Pub. No.: US 2015/0021576 A1
(43) Pub. Date: Jan. 22, 2015

(54) MATERIAL FOR AN ORGANIC ELECTROLUMINESCENCE DEVICE AND ORGANIC ELECTROLUMINESCENCE DEVICE INCLUDING THE SAME

(71) Applicant: SAMSUNG DISPLAY CO., LTD., Yongin-City (KR)

(72) Inventor: Hiroaki ITOI, Yokohama (JP)

(21) Appl. No.: 14/332,764

(22) Filed: Jul. 16, 2014

(30) Foreign Application Priority Data
Jul. 18, 2013 (JP) 2013-149661

(51) Int. Cl. H01L 51/00; H01L 51/52; H01L 51/50; C07D 209/86

(52) U.S. Cl. CPC H01L 51/0054; C07D 209/86; H01L 51/0072; H01L 51/0052; H01L 51/0058; H01L 51/5221; H01L 51/5012
USPC 257/40; 548/440

(57) ABSTRACT

A material for an electroluminescence device and an electroluminescence device including the same, the material being represented by following Formula 1:

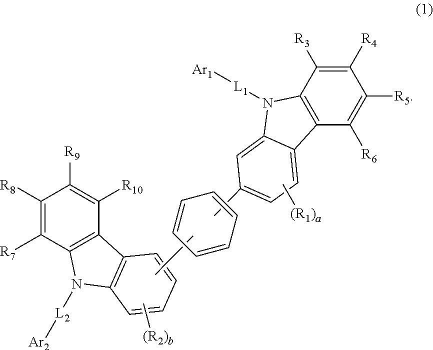

### CROSS-REFERENCE TO RELATED APPLICATION

Japanese Patent Application No. 2013-149661, filed on Jul. 18, 2013, in the Japanese Patent Office, and entitled: “Material For Organic Electroluminescence Device and Organic Electroluminescence Device Using the Same,” is incorporated by reference herein in its entirety.

### BACKGROUND

1. Field

Embodiments relate to a material for an organic electroluminescence device and an organic electroluminescence device including the same.

2. Description of the Related Art

Organic electroluminescence (EL) displays, which are one type of image display, have been considered. Unlike a liquid crystal display or the like, the organic EL display is a self-luminescent display that recombines holes and electrons (injected from a positive electrode and a negative electrode) in an emission layer to thus emit lights from a light-emitting material (including an organic compound of the emission layer), thereby performing display.

An example of an organic electroluminescence device (hereinafter referred to as an organic EL device) may include an organic EL device that includes a positive electrode, a hole transport layer on the positive electrode, an emission layer on the hole transport layer, an electron transport layer on the emission layer, and a negative electrode on the electron transport layer. Holes injected from the positive electrode may be injected into the emission layer via the hole transport layer. Meanwhile, electrons may be injected from the negative electrode, and then injected into the emission layer via the electron transport layer. The holes and the electrons injected into the emission layer may be recombined to generate excitons within the emission layer. The organic EL device may emit light generated by the radiation and deactivation of the excitons.

### SUMMARY

Embodiments are directed to a material for an organic electroluminescence device and an organic electroluminescence device including the same.

The embodiments may be realized by providing a material for an electroluminescence device, the material being represented by following Formula 1:

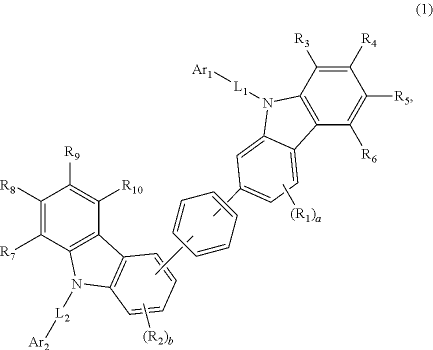

wherein, in Formula 1 Ar₁ is an aryl group having 6 to 30 carbon atoms, a heteroaryl group having 5 to 30 carbon atoms, or an alkyl group having 1 to 15 carbon atoms, Ar₂ is a condensed ring-containing group having 6 to 30 carbon atoms or a condensed ring-containing group having a carbon atom and a nitrogen atom, Ar₁ and Ar₂ are different from each other, R₁ to R₁₀ are each independently an aryl group having 6 to 30 carbon atoms, a heteroaryl group having 5 to 30 carbon atoms, an alkyl group having 1 to 15 carbon atoms, a halogen atom, a hydrogen, or a deuterium, or two or more of adjacent ones of R₁ to R₁₀ are combined to form a saturated or unsaturated ring, a and b are each independently an integer of 0 to 3, and L₁ and L₂ are each independently a single bond or a divalent connecting group having 4 or more carbon atoms.

Two or more of adjacent ones of R₁ to R₁₀ may be combined to form a saturated or unsaturated ring, other than an aromatic ring including R₁ and R₆ or R₂ and R₁₀.

The embodiments may be realized by providing a material for an electroluminescence device, the material being represented by following Formula 2:

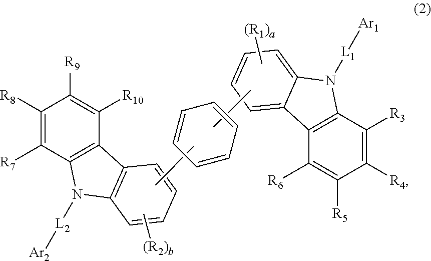

wherein, in Formula 2 Ar₁ is an aryl group having 6 to 30 carbon atoms, a heteroaryl group having 5 to 30 carbon atoms, or an alkyl group having 1 to 15 carbon atoms, Ar₂ is a condensed ring-containing group having 6 to 30 carbon atoms or a condensed ring-containing group having a carbon atom and a nitrogen atom, Ar₁ and Ar₂ are different from each other, R₁ to R₁₀ are each independently an aryl group having 6 to 30 carbon atoms, a heteroaryl group having 5 to 30 carbon atoms, an alkyl group having 1 to 15 carbon atoms, a halogen atom, a hydrogen, or a deuterium, or two or more of adjacent ones of R₁ to R₁₀ are combined to form a saturated or unsaturated ring, a and b are each independently an integer of 0 to 3, and L₁ and L₂ are each independently a single bond or a divalent connecting group having 4 or more carbon atoms.

Two or more of adjacent ones of R₁ to R₁₀ may be combined to form a saturated or unsaturated ring, other than an aromatic ring including R₁ and R₆ or R₂ and R₁₀.

The embodiments may be realized by providing a material for an electroluminescence device, the material being represented by following Formula 3:

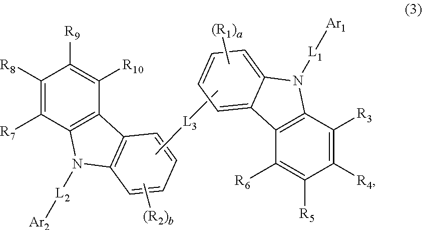

wherein, in Formula 3 Ar₁ is an aryl group having 6 to 30 carbon atoms, a heteroaryl group having 5 to 30 carbon atoms, or an alkyl group having 1 to 15 carbon atoms, Ar₂ is a condensed ring-containing group having 6 to 30 carbon atoms or a condensed ring-containing group having a carbon atom and a nitrogen atom, Ar₁ and Ar₂ are different from each other, R₁ to R₁₀ are each independently an aryl group having 6 to 30 carbon atoms, a heteroaryl group having 5 to 30 carbon atoms, an alkyl group having 1 to 15 carbon atoms, a halogen atom, a hydrogen, or a deuterium, or two or more of adjacent ones of R₁ to R₁₀ are combined to form a saturated or unsaturated ring, a and b are each independently an integer of 0 to 3, L₁ and L₂ are each independently a single bond or a divalent connecting group having 4 or more carbon atoms, and L₃ is a divalent connecting group having 4 or more carbon atoms.

Two or more of adjacent ones of R₁ to R₁₀ may be combined to form a saturated or unsaturated ring, other than an aromatic ring including R₁ and R₆ or R₂ and R₁₀.

The embodiments may be realized by providing an electroluminescence device including an emission layer; and a positive electrode, wherein the material for an electroluminescence device according to an embodiment is in the emission layer or in another between an emission layer and the positive electrode.

### BRIEF DESCRIPTION OF THE DRAWING

Features will be apparent to those of skill in the art by describing in detail exemplary embodiments with reference to the attached drawing in which:

FIG. 1 illustrates the structure of an organic EL device according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

An organic EL device having high efficiency and long life may be realized by including a material for an organic EL device that includes a condensed ring-containing group in or on a carbazole unit or moiety. For example, such efficiency and long life may not be realized when using an aromatic amine compound having low electron resistance.

The material for an organic EL device according to an embodiment may include a carbazole derivative, e.g., a carbazole-containing compound including two carbazole moieties, and a condensed ring-containing group in or on one of the carbazole moieties, as represented by the following Formula 3.

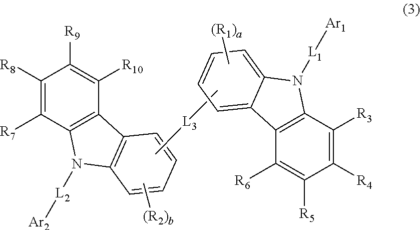

In Formula 3, Ar₁ may be an aryl group having 6 to 30 carbon atoms, a heteroaryl group having 5 to 30 carbon atoms, or an alkyl group having 1 to 15 carbon atoms. In an implementation, Ar₁ may be a halogen-containing group. Ar₂ may be a condensed ring-containing group having, e.g., a carbon atom skeleton of, 6 to 30 carbon atoms, or a condensed ring-containing group having a carbon atom and a nitrogen atom. Ar₁ and Ar₂ may be different from each other. For example, only one carbazole moiety of the compound may include a condensed ring-containing group bonded thereto. In an implementation, Ar₂ may include, e.g., a triphenylenyl group, a phenanthrenyl group, a naphthyl group, or the like. In an implementation, the material according to an embodiment may be an asymmetrical compound.

In Formula 3, R₁ to R₁₀ may each independently be an aryl group having 6 to 30 carbon atoms, a heteroaryl group having 5 to 30 carbon atoms, an alkyl group having 1 to 15 carbon atoms, a halogen atom, a hydrogen, or a deuterium. a and b may each independently be an integer of 0 to 3. L₁ and L₂ may each independently be, e.g., a single bond, a divalent connecting group having 4 or more single bonded carbon atoms, or a divalent connecting group having 4 or more carbon atoms. L₃ may be a divalent connecting group having 4 or more carbon atoms.

In an implementation, when introducing or including a condensed ring-containing group on a carbazole moiety, hole transporting properties and electron resistance may be improved, and a hole transport layer having high efficiency and long life may be formed in an organic EL device.

In Formula 3, two or more adjacent ones of R₁ to R₁₀ may be combined or fused to form a saturated or unsaturated ring. For example, two adjacent ones of R₁ to R₁₀ may be fused to form a ring.

For example, the material for an organic EL device according to an embodiment may include two carbazole moieties combined or bound through a phenylene group. In an implementation, the material according to an embodiment may be represented by the following Formula 2. In an implementation, in the material represented by Formula 2, a triphenylene moiety may be included on one of the carbazole moieties.

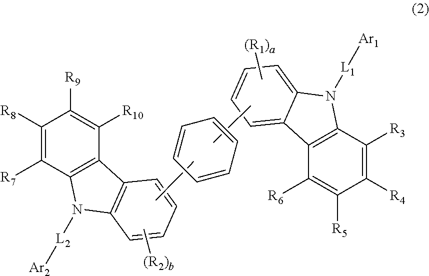

In Formula 2, Ar₁ may be an aryl group having 6 to 30 carbon atoms, a heteroaryl group having 5 to 30 carbon atoms, or an alkyl group having 1 to 15 carbon atoms. Ar₂ may be a condensed ring-containing group having, e.g., a carbon atom skeleton of, 6 to 30 carbon atoms or a condensed ring-containing group having a carbon atom and a nitrogen atom. Ar₁ and Ar₂ may be different from each other. In an implementation, only one of the carbazole moieties may include the condensed ring-containing group thereon or bonded thereto.

In Formula 2, R₁ to R₁₀ may each independently be an aryl group having 6 to 30 carbon atoms, a heteroaryl group having 5 to 30 carbon atoms, an alkyl group having 1 to 15 carbon atoms, a halogen atom, a hydrogen, or a deuterium. a and b may each independently be an integer of 0 to 3. L₁ and L₂ may each independently be a single bond, a divalent connecting group having 4 or more single bonded carbon atoms, or a divalent connecting group having 4 or more carbon atoms.

The material for an organic EL device according to an embodiment may include two carbazole moieties combined or bonded through a phenylene group and may include a condensed ring-containing group on one of the carbazole moieties, a degree of conjugation of the whole compound may be appropriately high, and an improvement of the hole transport properties may be expected.

In an implementation, the material for an organic EL device according to an embodiment may include two carbazole moieties combined or bonded through a phenylene group. For example, the material may be represented by the following Formula 1, in which one carbazole moiety may include a condensed ring-containing group, and may include the phenylene group at position 2 of the carbazole moiety.

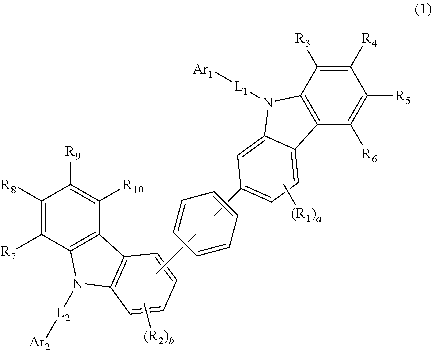

In Formula 1, Ar₁ may be an aryl group having 6 to 30 carbon atoms, a heteroaryl group having 5 to 30 carbon atoms, or an alkyl group having 1 to 15 carbon atoms. Ar₂ may be a condensed ring-containing group having, e.g., a carbon atom skeleton of, 6 to 30 carbon atoms or a condensed ring-containing group having a carbon atom and a nitrogen atom. Ar₁ and Ar₂ may be different from each other. In an implementation, only one carbazole moiety may include a condensed ring-containing group thereon or bonded thereto.

In Formula 1, R₁ to R₁₀ may each independently be an aryl group having 6 to 30 carbon atoms, a heteroaryl group having 5 to 30 carbon atoms, an alkyl group having 1 to 15 carbon atoms, a halogen atom, a hydrogen, or a deuterium. a and b may each independently be an integer of 0 to 3. L₁ and L₂ may each independently be a single bond, a divalent connecting group having 4 or more single bonded carbon atoms, or a divalent connecting group having 4 or more carbon atoms.

In an implementation, the material for an organic EL device may be a carbazole derivative (e.g., a carbazole group-containing compound) in which two carbazole moieties are combined or bonded through a phenylene group, and one carbazole moiety (including a condensed ring-containing group thereon) may be bound to the phenylene group at position 2 of the carbazole moiety, thereby lowering the energy level of HOMO and controlling hole transporting properties.

In an implementation, the material for an organic EL device according to an embodiment may be represented by one of the following Compounds 7 to 12.

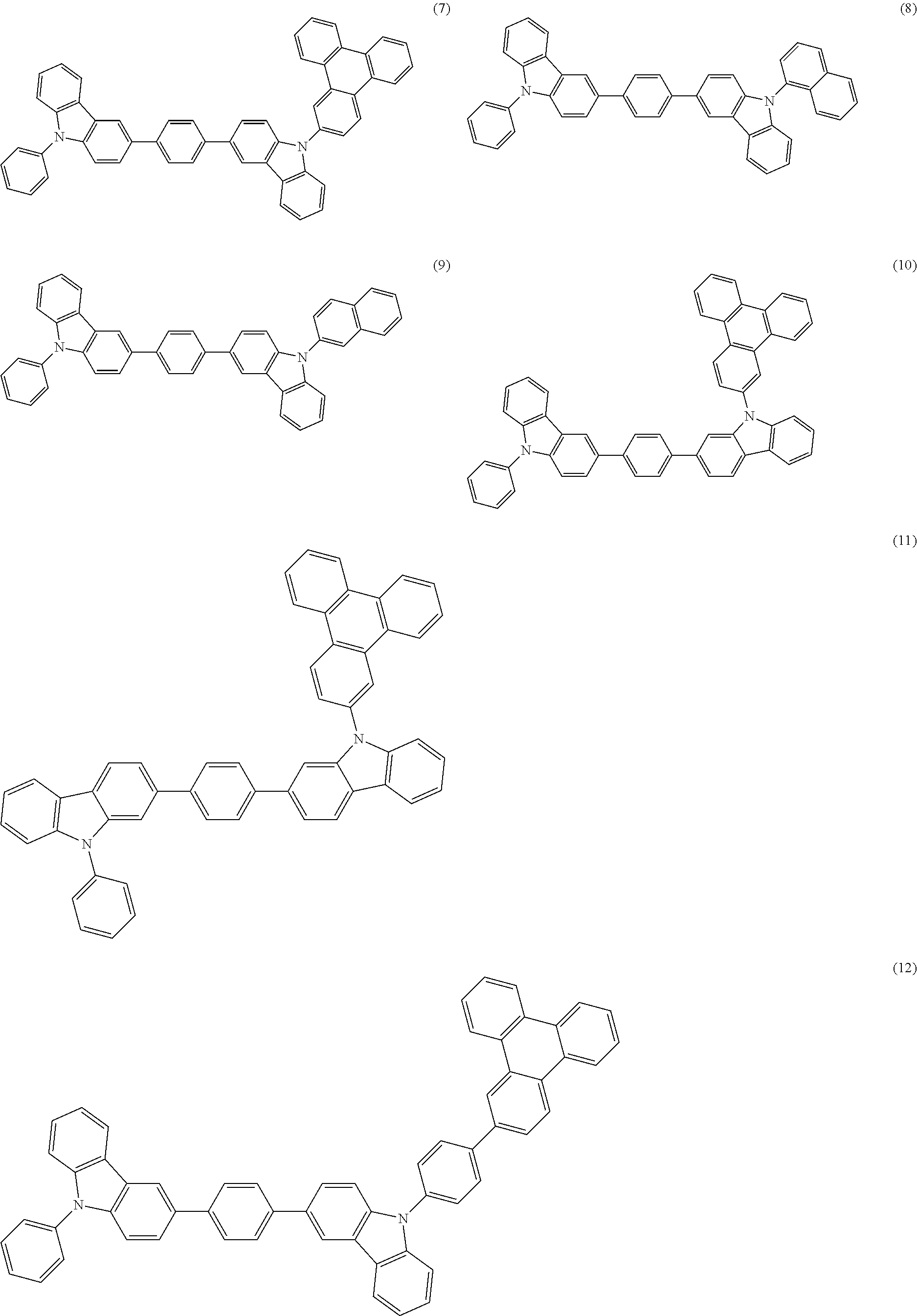

In an implementation, the material for an organic EL device according to an embodiment may be represented by one of the following Compounds 13 to 18.

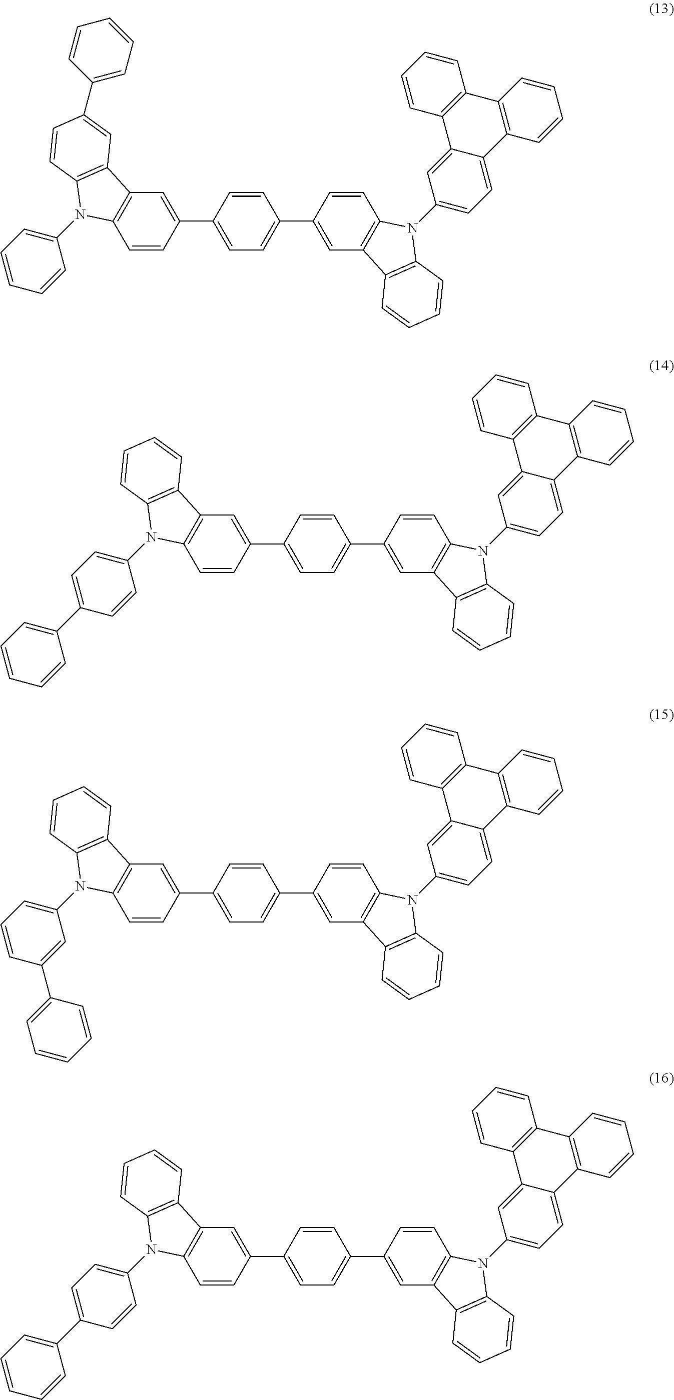 [SMILES: c1ccc(-c2ccc3c(c2)c2cc(-c4ccc(-c5ccc6c(c5)c5ccccc5n6-c5ccc6c7ccccc7c7ccccc7c6c5)cc4)ccc2n3-c2ccccc2)cc1 ; c1ccc(-c2ccc(-n3c4ccccc4c4cc(-c5ccc(-c6ccc7c(c6)c6ccccc6n7-c6ccc7c8ccccc8c8ccccc8c7c6)cc5)ccc43)cc2)cc1 ; c1ccc(-c2cccc(-n3c4ccccc4c4cc(-c5ccc(-c6ccc7c(c6)c6ccccc6n7-c6ccc7c8ccccc8c8ccccc8c7c6)cc5)ccc43)c2)cc1 ; c1ccc(-c2ccc(-n3c4ccccc4c4cc(-c5ccc(-c6ccc7c(c6)c6ccccc6n7-c6ccc7c8ccccc8c8ccccc8c7c6)cc5)ccc43)cc2)cc1]

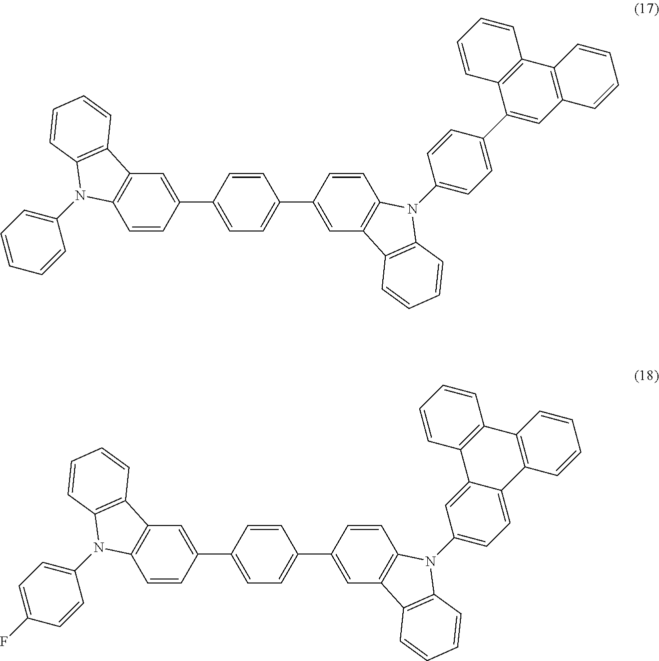 [SMILES: c1ccc(-n2c3ccccc3c3cc(-c4ccc(-c5ccc6c(c5)c5ccccc5n6-c5ccc(-c6cc7ccccc7c7ccccc67)cc5)cc4)ccc32)cc1 ; Fc1ccc(-n2c3ccccc3c3cc(-c4ccc(-c5ccc6c(c5)c5ccccc5n6-c5ccc6c7ccccc7c7ccccc7c6c5)cc4)ccc32)cc1]

In an implementation, the material for an organic EL device according to an embodiment may be represented by one of the following Compounds 19 to 24.

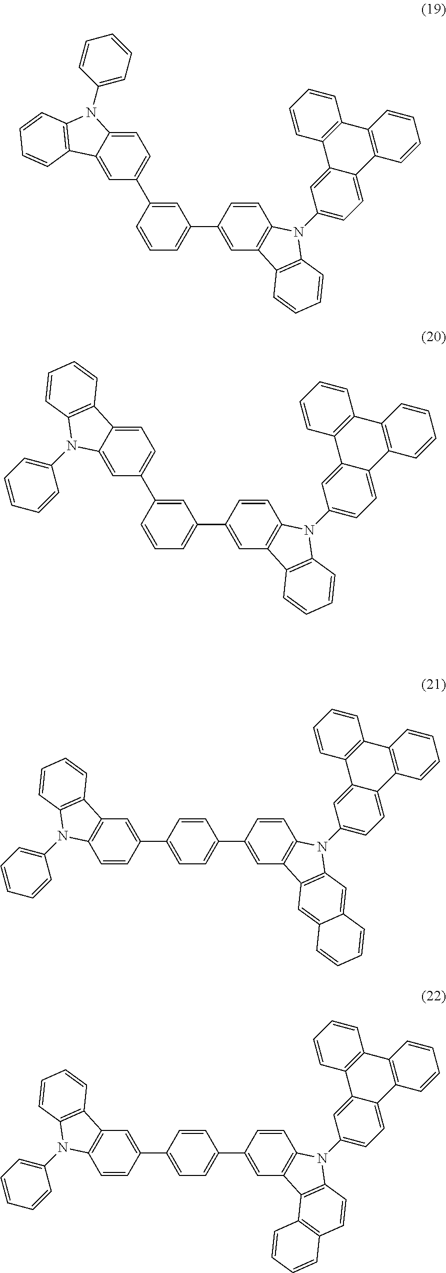 [SMILES: c1ccc(-n2c3ccccc3c3cc(-c4cccc(-c5ccc6c(c5)c5ccccc5n6-c5ccc6c7ccccc7c7ccccc7c6c5)c4)ccc32)cc1 ; c1ccc(-n2c3ccccc3c3ccc(-c4cccc(-c5ccc6c(c5)c5ccccc5n6-c5ccc6c7ccccc7c7ccccc7c6c5)c4)cc32)cc1 ; c1ccc(-n2c3ccccc3c3cc(-c4ccc(-c5ccc6c(c5)c5cc7ccccc7cc5n6-c5ccc6c7ccccc7c7ccccc7c6c5)cc4)ccc32)cc1 ; c1ccc(-n2c3ccccc3c3cc(-c4ccc(-c5ccc6c(c5)c5c7ccccc7ccc5n6-c5ccc6c7ccccc7c7ccccc7c6c5)cc4)ccc32)cc1]

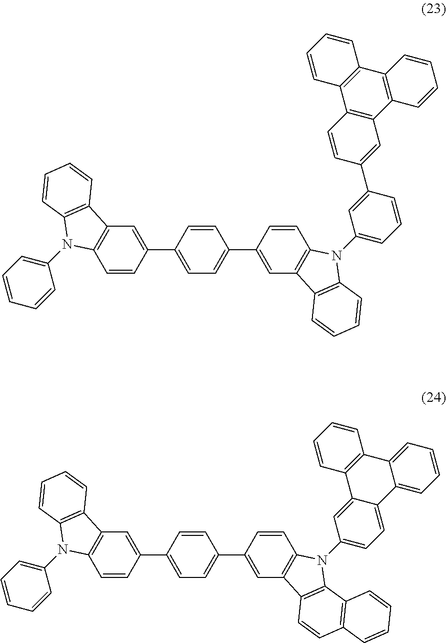 [SMILES: c1ccc(-n2c3ccccc3c3cc(-c4ccc(-c5ccc6c(c5)c5ccccc5n6-c5cccc(-c6ccc7c8ccccc8c8ccccc8c7c6)c5)cc4)ccc32)cc1 ; c1ccc(-n2c3ccccc3c3cc(-c4ccc(-c5ccc6c(c5)c5ccc7ccccc7c5n6-c5ccc6c7ccccc7c7ccccc7c6c5)cc4)ccc32)cc1]

In an implementation, the material for an organic EL device according to an embodiment may be represented by one of the following Compounds 25 to 28.

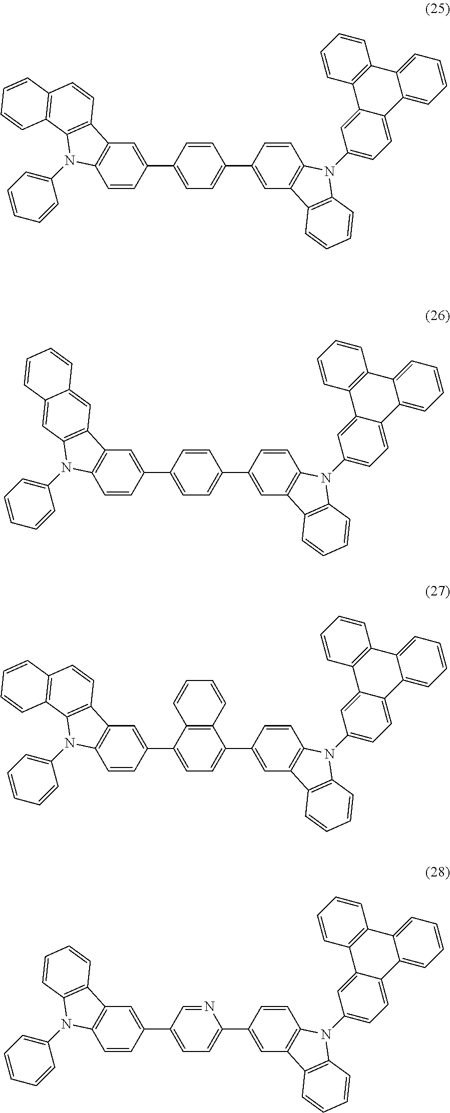 [SMILES: c1ccc(-n2c3ccc(-c4ccc(-c5ccc6c(c5)c5ccccc5n6-c5ccc6c7ccccc7c7ccccc7c6c5)cc4)cc3c3ccc4ccccc4c32)cc1 ; c1ccc(-n2c3ccc(-c4ccc(-c5ccc6c(c5)c5ccccc5n6-c5ccc6c7ccccc7c7ccccc7c6c5)cc4)cc3c3cc4ccccc4cc32)cc1 ; c1ccc(-n2c3ccc(-c4ccc(-c5ccc6c(c5)c5ccccc5n6-c5ccc6c7ccccc7c7ccccc7c6c5)c5ccccc45)cc3c3ccc4ccccc4c32)cc1 ; c1ccc(-n2c3ccccc3c3cc(-c4ccc(-c5ccc6c(c5)c5ccccc5n6-c5ccc6c7ccccc7c7ccccc7c6c5)nc4)ccc32)cc1]

In an implementation, the material for an organic EL device according to an embodiment may be suitably included in an emission layer of an organic EL device. In an implementation, the material for an organic EL device may be included in a layer (of stacked layers) between the emission layer and a positive electrode. Therefore, the hole transporting properties and the electron resistance may be improved, and the high efficiency and the long life of the organic EL device may be realized.

Organic EL Device

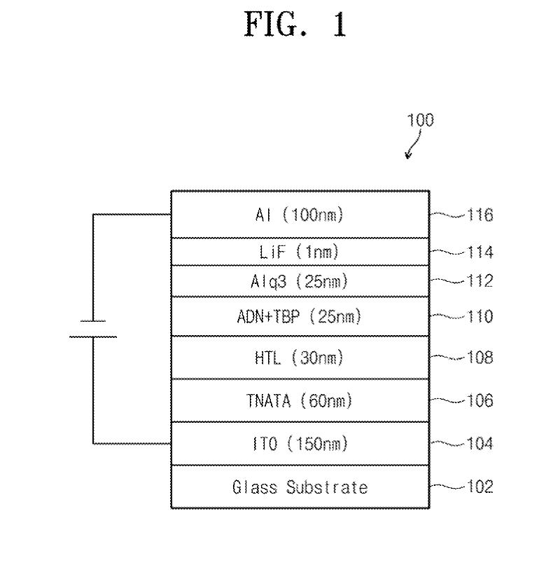

An organic EL device using or including the material for an organic EL device according to an embodiment will be explained. FIG. 1 illustrates a schematic diagram of the configuration of an organic EL device 100 according to an embodiment. The organic EL device 100 may include, e.g., a substrate 102, a positive electrode 104, a hole injection layer 106, a hole transport layer 108, an emission layer 110, an electron transport layer 112, an electron injection layer 114, and a negative electrode 116. In an implementation, the material for an organic EL device may be included in the emission layer of the organic EL device. In an implementation, the material for an organic EL device may be included in one of the layers between the emission layer and the positive electrode.

For example, an embodiment in which the material for an organic EL device is included in the hole transport layer 108 will be explained. The substrate 102 may include, e.g., a transparent glass substrate, a semiconductor substrate formed by using silicon, or the like, or a flexible substrate. The positive electrode 104 may be on the substrate 102, and may include, e.g., indium tin oxide (ITO), indium zinc oxide (IZO), or the like. The hole injection layer 106 may be on the positive electrode 104 and may include, e.g., 4,4′,4″-tris(N-1-naphthyl)-N-phenylamino)-triphenylamine (1-TNATA) or 4,4-bis[N,N-di(3-tolyl)amino]-3,3-dimethylbiphenyl (HMTPD), or the like. The hole transport layer 108 may be on the hole injection layer 106 and may be formed using the material for an organic EL device according to an embodiment. The emission layer 110 may be on the hole transport layer 108 and may be formed by doping tetra-t-butylperylene (TBP), or the like in a host material including, e.g., 9,10-di(2-naphthyl)anthracene (ADN). The electron transport layer 112 may be on the emission layer 110 and may include, e.g., tris(8-hydroxyquinolinato)aluminum (Alq₃), or the like. The electron injection layer 114 may be on the electron transport layer 112 and may include, e.g., lithium fluoride (LiF). The negative electrode 116 may be on the electron injection layer 114 and may include, e.g., a metal such as Al or a transparent material such as ITO, IZO, or the like. The above-described layers may be formed by selecting a suitable layer forming method, e.g., vacuum deposition, sputtering, various coatings, or the like.

In the organic EL device 100 according to an embodiment, a hole transport layer having high efficiency and long life may be formed by using the material for an organic EL device described above. In an implementation, the material for an organic EL device may be included in an organic EL apparatus of an active matrix using thin film transistors (TFT).

In an implementation, in the organic EL device 100 according to an embodiment, high efficiency and long life may be realized by using the material for an organic EL device in an emission layer or another layer between the emission layer and the positive electrode.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### EXAMPLES

### Preparation Method

A material for an organic EL device according to an inventive concept was synthesized by the following method of Reaction Scheme 1.

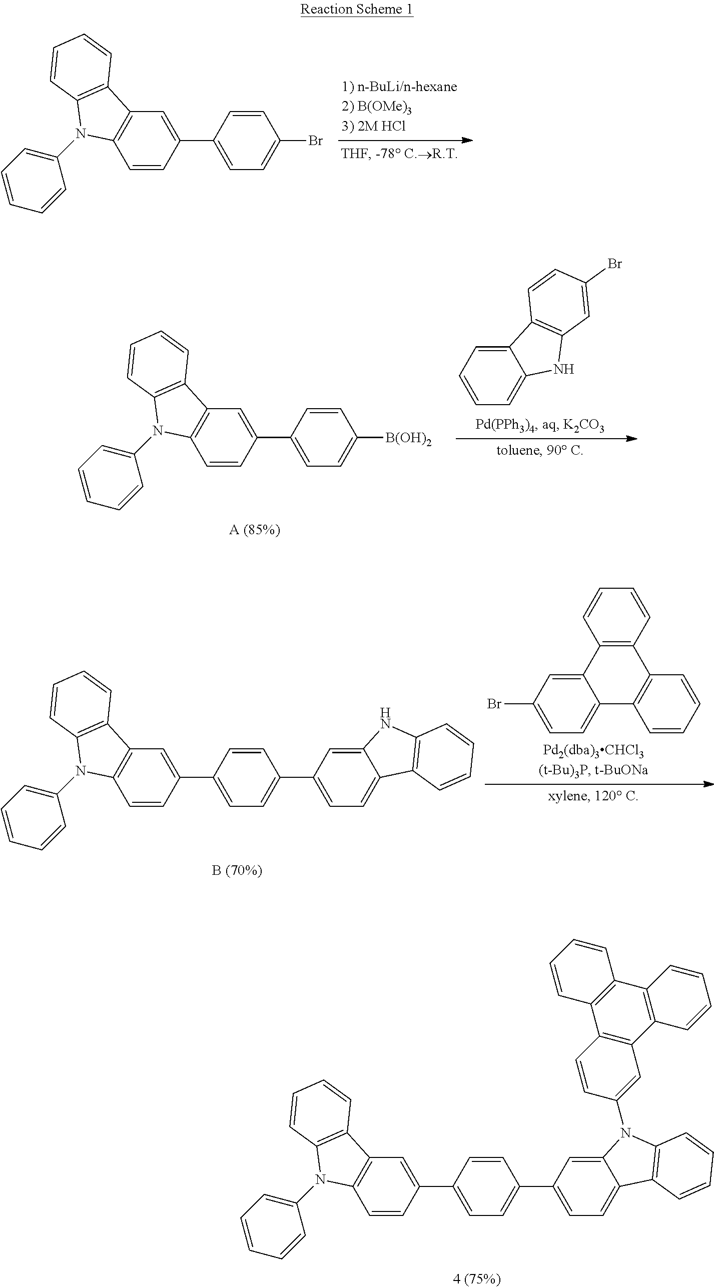 [SMILES: Brc1ccc(-c2ccc3c(c2)c2ccccc2n3-c2ccccc2)cc1 ; OB(O)c1ccc(-c2ccc3c(c2)c2ccccc2n3-c2ccccc2)cc1 ; Brc1ccc2c(c1)[nH]c1ccccc12 ; c1ccc(-n2c3ccccc3c3cc(-c4ccc(-c5ccc6c(c5)[nH]c5ccccc56)cc4)ccc32)cc1 ; Brc1ccc2c3ccccc3c3ccccc3c2c1 ; c1ccc(-n2c3ccccc3c3cc(-c4ccc(-c5ccc6c7ccccc7n(-c7ccc8c9ccccc9c9ccccc9c8c7)c6c5)cc4)ccc32)cc1]

(Synthesis of Compound A)

Under an argon atmosphere, 20.0 g of 3-(4-bromophenyl)-9-phenyl-9H-carbazole was added in a 1 L, four-necked flask, and stirred in 350 mL of a tetrahydrofuran (THF) solvent at −78° C. for 5 minutes. Then, 36.7 mL of 1.64 M n-butyl lithium (in n-hexane) was added thereto, followed by stirring at −78° C. for 1 hour. 11.2 mL of trimethoxyborane was added thereto, followed by stirring at ambient (e.g., room) temperature for 2 hours. After that, 200 mL of 2M aqueous hydrochloric acid solution was added, followed by stirring at ambient temperature for 3 hours. An organic layer was separated, and solvents were removed by distillation. Recrystallization was performed in a solvent system of ethyl acetate and hexane to obtain 15.69 g of Compound A as a white solid (yield 85%).

(Synthesis of Compound B)

Under an argon atmosphere, 14.0 g of Compound A, 10.4 g of 3-bromocarbazole, 3.12 g of tetrakis(triphenylphosphine) palladium (Pd(PPh₃)₄), 10.7 g of potassium carbonate (K₂CO₃), 80 mL of water, 30 mL of ethanol, and 400 mL of toluene were added in a 1 L, four-necked flask, and stirred at 90° C. for 4 hours. After cooling in air, an organic layer was separated, and solvents were removed by distillation. Then, recrystallization was performed using toluene to obtain 13.1 g of Compound B as a white solid (yield 70%).

(Synthesis of Compound 10)

Under an argon atmosphere, 9.70 g of Compound B, 6.76 g of 2-bromotriphenylene, 1.45 g of tris(dibenzylideneacetone)dipalladium(0) (Pd₂(dba)₃), 510 mg of tri-tert-butyl(phosphine) ((t-Bu)₃P), and 5.77 g of sodium tert-butoxide were added in a 500 mL three-necked flask and heated while stirring in 50 mL of a xylene solvent at 120° C. for 12 hours. After cooling in air, water was added thereto to separate an organic layer, and solvents were removed by distillation. The crude product thus obtained was separated by silica gel column chromatography (using a mixed solvent of dichloromethane and hexane), and recrystallization was performed using a mixed solvent of toluene and hexane to obtain 10.7 g of Compound 10 as a white solid (yield 75%).

(Identification of Compounds)

The identification of compounds was conducted by ¹H-NMR and FAB-MS.

(Identification of Compound A)

The chemical shift values of Compound A measured by the ¹H-NMR was 8.47 (d, 1H), 8.40 (d, 2H), 8.23 (d, 1H), 7.89 (d, 1H), 7.75-7.79 (m, 1H), 7.59-7.64 (m, 4H), 7.42-7.52 (m, 4H), 7.25-7.36 (m, 1H), 1.58 (s, 2H).

(Identification of Compound B)

The molecular weight of Compound B measured by FAB-MS was 484.

(Identification of Compound 10)

The chemical shift values of Compound 10 measured by the ¹H-NMR was 8.84-8.86 (m, 2H), 8.70-8.73 (m, 3H), 8.57 (d, 1H), 8.44 (q, 2H), 8.24-8.30 (m, 2H), 7.82-7.88 (m, 5H), 7.53-7.76 (m, 12H), 7.30-7.49 (m, 7H). In addition, the molecular weight of Compound 10 measured by FAB-MS was 710.

According to the above-described method, and with slight variations, compounds according to Examples 1 to 3 (corresponding with Compounds 10, 8, and 27) were prepared.

In addition, Comparative Compound 1 and Comparative Compound 2 were prepared.

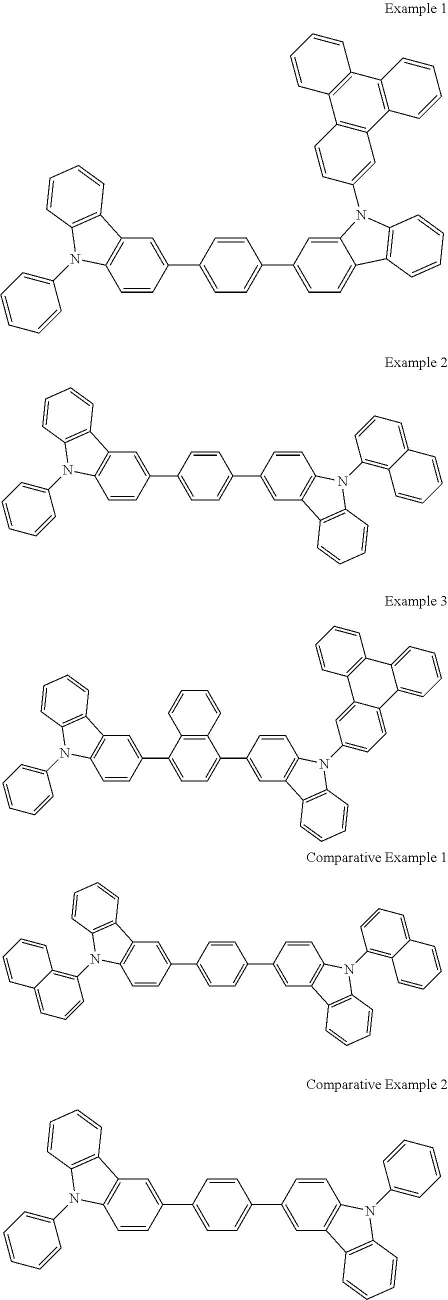 [SMILES: c1ccc(-n2c3ccccc3c3cc(-c4ccc(-c5ccc6c7ccccc7n(-c7ccc8c9ccccc9c9ccccc9c8c7)c6c5)cc4)ccc32)cc1 ; c1ccc(-n2c3ccccc3c3cc(-c4ccc(-c5ccc6c(c5)c5ccccc5n6-c5cccc6ccccc56)cc4)ccc32)cc1 ; c1ccc(-n2c3ccccc3c3cc(-c4ccc(-c5ccc6c(c5)c5ccccc5n6-c5ccc6c7ccccc7c7ccccc7c6c5)c5ccccc45)ccc32)cc1 ; c1ccc2c(-n3c4ccccc4c4cc(-c5ccc(-c6ccc7c(c6)c6ccccc6n7-c6cccc7ccccc67)cc5)ccc43)cccc2c1 ; c1ccc(-n2c3ccccc3c3cc(-c4ccc(-c5ccc6c(c5)c5ccccc5n6-c5ccccc5)cc4)ccc32)cc1]

Organic EL devices were manufactured by using the above compounds of Examples 1 to 3 and Comparative Examples 1 and 2 as hole transport materials. The substrate 102 was formed by using a transparent glass substrate, the positive electrode 104 was formed using ITO to a thickness of about 150 nm, the hole injection layer 106 having a thickness of about 60 nm was formed by using 2-TNATA, the hole transport layer 108 was formed using the above materials to a thickness of about 30 nm, the emission layer 110 was formed by doping about 3% of TBP in ADN to a thickness of about 25 nm, the electron transport layer 112 was formed using Alq₃ to a thickness of about 25 nm, the electron injection layer 114 was formed using LiF to a thickness of about 1 nm, and the negative electrode 116 was formed using Al to a thickness of about 100 nm.

With respect to the organic EL devices thus manufactured, the voltage, the current efficiency and the half life were evaluated. The current efficiency is the value at 10 mA/cm², and the half life is the time necessary for decreasing the luminance to half from the initial luminance of 1,000 cd/m². The results are illustrated in the following Table 1.

TABLE 1

| | Voltage | Current efficiency | Half life |
|---|---|---|---|
| | (V) | (cd/A) | (h) |
| Example 1 | 4.6 | 11.9 | 4,100 |
| Example 2 | 4.5 | 11.9 | 3,400 |
| Example 3 | 4.9 | 8.9 | 2,800 |
| Comparative Example 1 | 7.7 | 5.8 | 1,500 |
| Comparative Example 2 | 7.8 | 5.9 | 1,200 |

As may be seen in Table 1, organic EL devices including the compounds according to Examples 1 to 3 were driven at lower voltage, when compared to those including the compounds according to Comparative Examples 1 and 2. With respect to the current efficiency, the organic EL devices including the compounds according to Examples 1 to 3 had longer half life, when compared to those including the compounds according to Comparative Examples 1 and 2. The results were considered to be obtained by the improvement of the electron resistance through including a condensed ring-containing group. In addition, the organic EL devices including the compounds according to Examples 1 to 3 had quite high efficiency and long life, when compared to those including the compounds according to Comparative Examples 1 and 2, and the results were considered to be obtained because the compounds according to Examples 1 to 3 included the condensed ring-containing group on only one of the carbazole moieties, to make an asymmetric structure over the whole molecule, and affect molecular orientation or amorphous layer properties.

For example, the hole transport properties and the electron resistance were rapidly improved through including the triphenylene group on one of the carbazole moieties in the compound according to Example 1. In addition, a naphthyl group was included on one of the carbazole moieties in the compound according to Example 2. In this case, the half life was shorter, however the hole transport properties and the electron resistance were markedly improved, when compared to the results for Example 1. In the compound according to Example 3, a triphenylene group was included on one of the carbazole moieties. In this case, two carbazole moieties were bonded through the naphthalene group, and even though the electron resistance was improved when compared to the compounds including a phenylene group, the hole transport properties were deteriorated due to the polarities at both sides of two naphthalene groups.

In the material for an organic EL device according to an embodiment, a condensed ring-containing compound was bonded with one carbazole unit, the hole transporting properties and the electron resistance were improved, and a hole transport layer having high efficiency and long life that could not be obtained by using an aromatic amine compound having low electron resistance may be formed in an organic EL device.

By way of summation and review, in applying of the organic EL device to a display apparatus, high efficiency and long life of the organic EL device may be considered, and the normalization, stabilization and durability of a hole transport layer have been considered in an effort to realize the high efficiency and long life of the organic EL device.

As a hole transport material to be used in a hole transport layer, various compounds (e.g., a carbazole derivative, an aromatic amine compound, or the like) may be used. A carbazole derivative substituted with a condensed ring may be a favorable material for attaining the long life of a device. However, the aromatic amine compound may have low electron resistance, and it may be difficult for an organic EL device using the material to have sufficient emission life. An ideal organic EL device may have high efficiency, may be capable of being driven by a low voltage, and may have long emission life. For example, the emission efficiency of an organic EL device in a blue emission region may be lower when compared to a red emission region and a green emission region, thus, the emission efficiency may be improved.

The embodiments may provide a hole transport material for an organic electroluminescence device having high efficiency and long life.

The material for an organic EL device according to an embodiment may have improved hole transporting properties and electron resistance by, e.g., introducing a condensed ring in or on one carbazole moiety, and a hole transport layer having high efficiency and long life may be formed in an organic EL device.

The material for an organic EL device according to an embodiment may have improved hole transporting properties and electron resistance by, e.g., introducing a triphenylene unit in or on a carbazole-containing skeleton, and a hole transport layer having high efficiency and long life may be formed in an organic EL device.

According to an embodiment, a material for an organic EL device having high efficiency and long life, and an organic EL device including the same, may be provided.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A material for an electroluminescence device, the material being represented by following Formula 1: 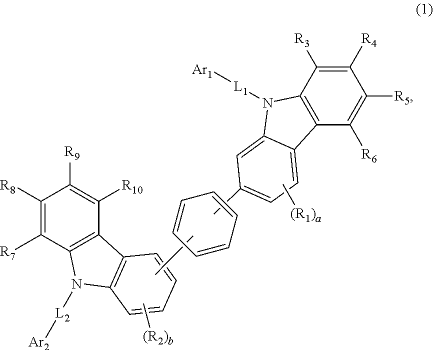
wherein, in Formula 1:
Ar₁ is an aryl group having 6 to 30 carbon atoms, a heteroaryl group having 5 to 30 carbon atoms, or an alkyl group having 1 to 15 carbon atoms,
Ar₂ is a condensed ring-containing group having 6 to 30 carbon atoms or a condensed ring-containing group having a carbon atom and a nitrogen atom,
Ar₁ and Ar₂ are different from each other,
R₁ to R₁₀ are each independently an aryl group having 6 to 30 carbon atoms, a heteroaryl group having 5 to 30 carbon atoms, an alkyl group having 1 to 15 carbon atoms, a halogen atom, a hydrogen, or a deuterium, or two or more of adjacent ones of R₁ to R₁₀ are combined to form a saturated or unsaturated ring,
a and b are each independently an integer of 0 to 3, and
L₁ and L₂ are each independently a single bond or a divalent connecting group having 4 or more carbon atoms.

2. A material for an electroluminescence device, the material being represented by following Formula 2: 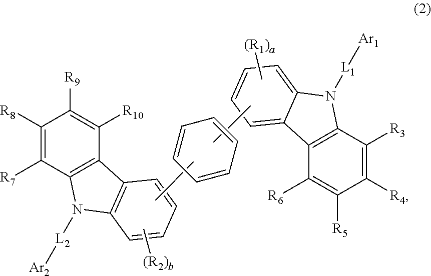
wherein, in Formula 2:
Ar₁ is an aryl group having 6 to 30 carbon atoms, a heteroaryl group having 5 to 30 carbon atoms, or an alkyl group having 1 to 15 carbon atoms,
Ar₂ is a condensed ring-containing group having 6 to 30 carbon atoms or a condensed ring-containing group having a carbon atom and a nitrogen atom,
Ar₁ and Ar₂ are different from each other,
R₁ to R₁₀ are each independently an aryl group having 6 to 30 carbon atoms, a heteroaryl group having 5 to 30 carbon atoms, an alkyl group having 1 to 15 carbon atoms, a halogen atom, a hydrogen, or a deuterium, or two or more of adjacent ones of R₁ to R₁₀ are combined to form a saturated or unsaturated ring,
a and b are each independently an integer of 0 to 3, and
L₁ and L₂ are each independently a single bond or a divalent connecting group having 4 or more carbon atoms.

3. A material for an electroluminescence device, the material being represented by following Formula 3: 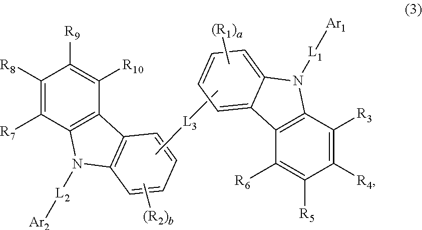
wherein, in Formula 3:
Ar₁ is an aryl group having 6 to 30 carbon atoms, a heteroaryl group having 5 to 30 carbon atoms, or an alkyl group having 1 to 15 carbon atoms,
Ar₂ is a condensed ring-containing group having 6 to 30 carbon atoms or a condensed ring-containing group having a carbon atom and a nitrogen atom,
Ar₁ and Ar₂ are different from each other,
R₁ to R₁₀ are each independently an aryl group having 6 to 30 carbon atoms, a heteroaryl group having 5 to 30 carbon atoms, an alkyl group having 1 to 15 carbon atoms, a halogen atom, a hydrogen, or a deuterium, or two or more of adjacent ones of R₁ to R₁₀ are combined to form a saturated or unsaturated ring,
a and b are each independently an integer of 0 to 3,
L₁ and L₂ are each independently a single bond or a divalent connecting group having 4 or more carbon atoms, and
L₃ is a divalent connecting group having 4 or more carbon atoms.

4. The material for an electroluminescence device as claimed in claim 1, wherein two or more of adjacent ones of R₁ to R₁₀ are combined to form a saturated or unsaturated ring, other than an aromatic ring including R₁ and R₆ or R₂ and R₁₀.

5. The material for an electroluminescence device as claimed in claim 2, wherein two or more of adjacent ones of R₁ to R₁₀ are combined to form a saturated or unsaturated ring, other than an aromatic ring including R₁ and R₆ or R₂ and R₁₀.

6. The material for an electroluminescence device as claimed in claim 3, wherein two or more of adjacent ones of R₁ to R₁₀ are combined to form a saturated or unsaturated ring, other than an aromatic ring including R₁ and R₆ or R₂ and R₁₀.

7. An electroluminescence device, comprising:
an emission layer; and
a positive electrode, wherein the material for an electroluminescence device as claimed in claim 1 is in the emission layer or in another between an emission layer and the positive electrode.

8. An electroluminescence device, comprising:
an emission layer; and
a positive electrode, wherein the material for an electroluminescence device as claimed in claim 2 is in the emission layer or in another between an emission layer and the positive electrode.

9. An electroluminescence device, comprising:
an emission layer; and
a positive electrode, wherein the material for an electroluminescence device as claimed in claim 3 is in the emission layer or in another between an emission layer and the positive electrode.
